(19) United States
(12) Patent Application Publication
(10) Pub. No.: US 2007/0154411 A1
(43) Pub. Date: Jul. 5, 2007

(54) Oral and dental care product

(76) Inventors: Adolf-Peter Barth, Haan (DE); Christian Kropf, Hilden (DE); Tilo Poth, Weinheim (DE)

Correspondence Address: PAUL & PAUL, 2000 MARKET STREET, PHILADELPHIA, PA, 19103-3229, US

(21) Appl. No.: 10/570,579

(22) PCT Filed: Aug. 24, 2004
(86) PCT No.: PCT/EP04/09438

§ 371 Date: Jan. 26, 2007

(30) Foreign Application Priority Data
Sep. 1, 2003 (DE) 103 40 543.7

(51) Int. Cl. A61K 8/96

(52) U.S. Cl. 424/50; 977/906

(57) ABSTRACT

The invention relates to oral and dental hygiene products comprising a) a composite material consisting of calcium salts which are slightly soluble in water, in the form of nanoparticulate primary particles having a length of from 5 to 150 nm and a cross section of from 2 to 50 nm and protein components selected from proteins, protein hydrolysates and protein hydrolysate derivatives, and b) 10 to 35% by weight of a cleaning agent mixture, the cleaning agent mixture comprising from 0.01 to 5% by weight of aluminum oxide polishing agent. The oral and dental hygiene products ensure a thorough cleaning of the teeth with simultaneous and long-lasting remineralization of the tooth surface.

The invention relates to oral and dental hygiene products having a content of composite materials and cleaning agents which, through specific selection of the cleaning agents, ensure an optimal cleaning with simultaneous remineralization of the tooth surface.

Oral and dental cleaning compositions which comprise a composite material composed of slightly soluble, nanoparticulate calcium salts and protein components in combination with cleaning and polishing agents have been disclosed. For example, WO 01/01930 A1 proposed a toothpaste based on approximately 10% abrasive silicas and 5% of a composite material. The effect of the composite material is based on the biomineralization of bone material and tooth material by closing dental lesions on the tooth surface. Abrasive substances by contrast are essential constituents of any toothpaste formulation and are responsible for cleaning the teeth and eliminating contaminants and deposits on the teeth by abrasion.

EP 786 245 A1 discloses numerous oral and dental hygiene products which relate to combination of a nanoparticulate hydroxyapatite and a cleaning agent. The hydroxyapatite is responsible for the remineralization of lesions on the tooth surface and for preventing caries. Aluminum hydroxides and calcium phosphates are used as cleaning agents, which are employed in high concentrations.

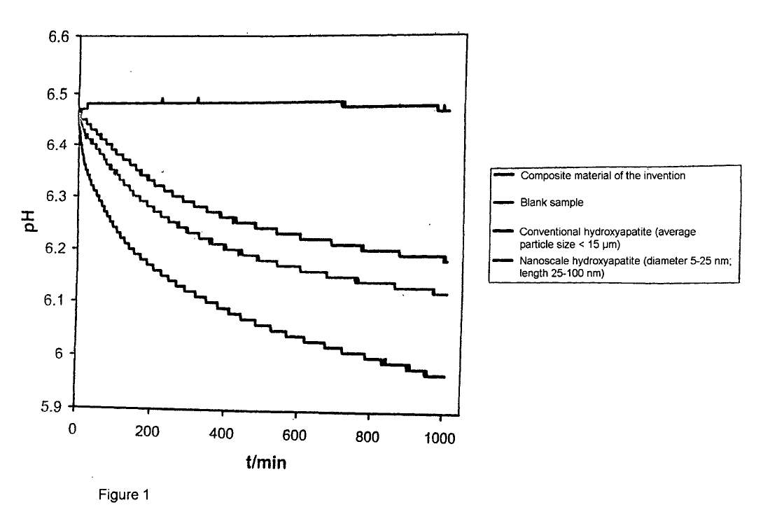

In practice, application of toothpaste formulations disclosed in WO 01/01930 A1 and EP 786 245 A1 has proved to be problematic, since the effect of the abrasive agent and of the remineralizing component are contrary to one another. Thus, it has been observed that, especially with the short time of about two to three minutes for which a toothpaste acts in the mouth, the depot effect of the remineralizing component was limited by the cleaning and polishing effect of the abrasive agent. Evidently, the deposited remineralizing component was to a large extent removed again by the abrasive substances in the cleaning process. It is therefore essential that the remineralizing component has very good remineralizing properties, so that the remineralizing effect during the cleaning process is still satisfactory. The comparison of various remineralizing components such as conventional hydroxyapatite, nanoscale hydroxyapatite and the composite material of the invention in FIG. 1 shows clear differences in respect of their remineralizing properties.

FIG. 1 shows the time course of the pH measured in a 0.1% strength dispersion of the respective material in simulated saliva at 37° C. The simulated saliva used for the present investigation consists of aqueous solution of 14 mM Na⁺, 4.7 mM PO₄³⁻, 21 mM K⁺, 30 mM Cl⁻, 1.8 mM Ca²⁺ and thus is supersaturated in calcium phosphate. The pH was followed using a pH electrode (Inlab 410, Mettler Toledo: meter: Consort, Multi Parameter Analyzer C833).

The change in pH with time results from the formation of hydroxyapatite from the saliva (i.e. the remineralizing effect), which satisfies the following equation:
10 CaCl₂+6 Na₂HPO₄+2 H₂O→Ca₁₀(OH)₂(PO₄)₆+12 NaCl+8 HCl

Acid is liberated in this reaction, and the pH falls, to a greater extent as the remineralizing effect of the tested material becomes better.

The substantially steeper decline in the pH in the case of the composite material compared with conventional hydroxypapatite or nanoscale hydroxyapatite shows the superior remineralizing properties of the composite material. It is thus outstandingly suitable as remineralizing component in a toothpaste formulation in which the depot effect of the remineralizing component is always opposed by the cleaning and polishing effect of an abrasive agent.

Besides remineralization, the oral and dental hygiene products of the invention are, however, also intended to show an optimal cleaning efficiency, i.e. that cleaning agents cannot be dispensed with.

However, both the amount and the nature and composition of the cleaning agent has an influence on the remineralization, which is why the object of the present invention was to produce an oral and dental cleaning composition which has a high cleaning efficiency and at the same time shows a good depot effect of the remineralizing component.

Surprisingly, an oral and dental hygiene product which has an improved cleaning effect (compared with the examples of WO 01/01930) while having a constant depot effect of the composite material has been produced by combining the composite material with a cleaning agent mixture which comprises a minimum content of aluminium oxide. This ensures a particularly thorough cleaning of the tooth surface with simultaneous remineralization.

The compositions of the invention are additionally distinguished by a repair effect. Irregularities and damage of the enamel, for example scratches on the enamel through mechanical action, are smoothed by being “filled in” by hydroxyapatite. Besides the repair of damaged enamel surfaces, this leads to an esthetically attractive surface. In addition, pain-sensitive teeth are prevented so that the compositions of the invention make sensitive cleaning possible.

The present invention therefore relates to an oral and dental hygiene product based on
a) a composite material comprising
   calcium salts which are slightly soluble in water, in the form of nanoparticulate primary particles having a length of from 5 to 150 nm and a cross section of from 2 to 50 nm and
   protein components selected from proteins, protein hydrolysates and protein hydrolysate derivatives, and
b) 10 to 35% by weight of a cleaning body mixture

where the cleaning body mixture comprises from 0.01 to 5% by weight of aluminum oxide polishing agent.

The term “composite materials” means, composites which include the components mentioned under a) and represent microscopically heterogeneous aggregates which, however, appear macroscopically homogeneous and in which the primary particles of the calcium salts are associated with the framework of the protein component. The proportion of the protein component(s) in the composite materials is between 0.1 and 60% by weight, but preferably between 2 and 50% by weight, in particular between 20 and 50% by weight, based on the total weight of the composite materials.

Primary particles mean the crystallites, i.e. the individual crystallites of said calcium salts. The particle diameter is to be understood here to mean the diameter of the particle, in the direction of its greatest longitudinal extent The average particle diameter means a value averaged over the total amount of the composite. Determination of the particle diameters can be made by methods familiar to the skilled worker, for example by Scherrer analysis from X-ray diffractometry investigations.

The average particle diameter of the nanoparticulate primary particles is preferably in the range from 5 to 150 nm, and they are particularly preferably in the form of rod-like particles with a thickness in the range from 2 to 50 nm, in particular 3 to 8 nm, and a length in the range from 5 to 150 nm, in particular 10 to 40 nm. Thickness means here the smallest diameter of the rods, and length their greatest diameter.

In a preferred embodiment of the invention, the nanoparticulate primary particles exhibit rod-like crystals with an average length-to-breadth ratio of the crystal of from 3 to ≦5. in particular of about 4. The average length-to-breadth ratio means according to the invention that a majority of the crystals have a length-to-breadth ratio in the stated range.

The length-to-breadth ratio is likewise determined by the X-ray diffraction method.

The spatial structure of the composite materials of the invention composed of a protein component and of the slightly soluble nanoparticulate calcium salts is clear from the example of the TE micrograph depicted in FIG. 1 of a composite material composed of hydroxyapatite and type A gelatin (200 000× magnification; 1 cm in the figure corresponds to 40 nm). The high molecular weight protein component, which assumes a three-dimensional structure which is substantially determined by its amino acid sequence, has the rod-like hydroxyapatite nanoparticles deposited thereon, and thus the nanoparticles to a certain extent form an image of the spatial structure of the protein component. This is clear from FIG. 2, which shows a TE micrograph of the type A gelatin framework of the same composite material after the hydroxyapatite has been dissolved out using a solution of ethylenediaminetetraacetate (56 000× magnification; 1.1 cm in the figure corresponds to 200 nm). The way in which the inorganic particles are deposited on the basic framework of the protein component is determined by the primary structure (amino acid sequence) and, depending on the nature of the protein component, its secondary, tertiary and quaternary structure. It has surprisingly been found that the spatial distribution and the quantitative extent of the deposition of the inorganic nanoparticles on the protein component can be influenced by the nature and amount of the amino acids present in the protein component, and thus by the selection of the protein components. Thus, for example, a particularly high loading with the slightly soluble calcium salt can be achieved by selecting protein components which are rich in the amino acids, aspartic acid, glutamic acid or cysteine. It is additionally possible to achieve a loading, which is spatially structured in a particular way, of the protein component with the slightly soluble calcium salt depending on the spatial distribution of these amino acids in the protein framework.

The composite materials of the invention are thus structured composite materials in contrast to the composite of hydroxyapatite and collagen which is described by R. Z. Wang et al. and in which uniformly distributed hydroxyapatite nanoparticles are present. A further substantial difference between the subject matter of the present invention and the prior art is the size and morphology of the inorganic component. The hydroxyapatite particles present in the hydroxyapatite-collagen composite described by R. Z. Wang et al. have a size of 2-10 nm. Hydroxyapatite particles in this size range are to be assigned to the range of amorphous or partially X-ray amorphous substances.

It has surprisingly been possible with the present invention to generate composite materials with crystalline inorganic nanoparticles in which the nanoparticles have a crystalline morphology which can be recognized clearly under the microscope. FIG. 1 shows the rod-like structure of the inorganic nanoparticles. It has further been found that the structured composite materials of the invention by contrast to the prior art lead to a particularly effective biomineralization process. It is assumed that this is connected to the microstructure of the composite material and in particular the size and morphology of the calcium salt crystals. Thus, it is assumed that the long axis of the calcium salt nanoparticles represents a preferred direction for further crystal growth during biomineralization.

Salts which are slightly soluble in water are intended to mean those whose solubility at 20° C. is less than 1 g/l. Preferably suitable calcium salts are calcium hydroxyphosphate (Ca₅[OH(PO₄)₃]) or hydroxyapatite, calcium fluorophosphate (Ca₅[F(PO₄)₃]) or fluoroapatite, fluorine-doped hydroxyapatite of the general composition Ca₅(PO₄)₃(OH,F) and calcium fluoride (Ca F₂) or fluorite (fluorspar); hydroxyapatite and/or fluoroapatite are particularly preferred.

The composite materials of the invention may comprise as calcium salt one salt or else a plurality of salts in a mixture selected from the group of phosphates, fluorides and fluorophosphates, which may optionally additionally comprise hydroxyl and/or carbonate groups, in the mixture.

Proteins suitable for the purposes of the present invention are in principle all proteins irrespective of their origin or their preparation. Examples of proteins of animal origin are keratin, elastin, collagen, fibrin, albumin, casein, whey protein, placental protein. From these, preference is given according to the invention to collagen, keratin, casein, whey protein, proteins of plant origin such as, for example, wheat and wheatgerm protein, rice protein, soybean protein, oat protein, pea protein, potato protein, almond protein and yeast protein may likewise be preferred according to the invention.

Protein hydrolysates mean for the purposes of the present invention degradation products of proteins such as, for example, collagen, elastin, casein, keratin, almond, potato, wheat, rice and soybean protein which are obtained by acidic, alkaline and/or enzymatic hydrolysis of the proteins themselves or their degradation products such as, for example, gelatin. Suitable for the enzymatic degradation are all enzymes having hydrolytic activity, such as, for example, alkaline proteases. Further suitable enzymes and enzymatic hydrolysis methods are described for example in K. Drauz and H. Waldmann, Enzyme Catalysis in Organic Synthesis, VCH-Verlag, Weinheim 1975. In the degradation, the proteins are split into smaller subunits, and the degradation may proceed via the stages of polypeptides to oligopeptides and on to the individual amino acids. Protein hydrolysates with little degradation include for example the gelatin which is preferred for the purposes of the present invention and which may have molecular masses in the range from 15 000 to 250 000 D. Gelatin is a polypeptide which is obtained principally by hydrolysis of collagen under acidic (type A gelatin) or alkaline (type B gelatin) conditions. The gel strength of the gelatin is proportional to its molecular weight, i.e. gelatin which has been hydrolyzed to a greater extent yields a solution of lower viscosity. The gel strength of gelatin is indicated in Bloom numbers. The polymer size is greatly reduced in the enzymatic cleavage of gelatin, leading to very low Bloom numbers.

Further preferred as protein hydrolysates for the purposes of the present invention are the protein hydrolysates used in cosmetics and having an average molecular weight in the range from 600 to 4000, particularly preferably from 2000 to 3500. Reviews of the preparation and use of protein hydrolysates have been published for example by G. Schuster and A. Domsch in Seifen Öle Fette Wachse 108 (1982) 177 and Cosm.Toil. 99, (1984) 63, by H. W. Steisslinger in Parf.Kosm. 72, (1991) 556 and F. Aurich et al. in Tens.Surf.Det. 29 (1992) 389. Preferably employed according to the invention are protein hydrolysates from collagen, keratin, casein and plant proteins, for example those based on wheat gluten or rice protein, the preparation of which is described in the two German patents DE 19502167 C1 and DE 19502168 C1 (Henkel).

Protein hydrolysate derivatives mean for the purposes of the present invention chemically and/or chemoenzymatically modified protein hydrolysates such as, for example, the compounds known under the INCI names sodium cocoyl hydrolyzed wheat protein, laurdimonium hydroxypropyl hydrolyzed wheat protein, potassium cocoyl hydrolyzed collagen, potassium undecylenoyl hydrolyzed collagen and laurdimonium hydroxypropyl hydrolyzed collagen. Preferably employed according to the invention are derivatives of protein hydrolysates of collagen, keratin and casein, and plant protein hydrolysates such as, for example, sodium cocoyl hydrolyzed wheat protein or laurdimonium hydroxypropyl hydrolyzed wheat protein.

Further examples of protein hydrolysates and protein hydrolysate derivatives which fall within the framework of the present invention are described in CTFA 1997 International Buyers' Guide, John A. Wenninger et al. (Ed.), The Cosmetic, Toiletry, and Fragrance Association, Washington DC 1997, 686-688.

The protein component can in each of the composite materials of the invention be formed by one or more substances selected from the group of proteins, protein hydrolysates and protein hydrolysate derivatives.

Preferred protein components are all structure-forming proteins, protein hydrolysates and protein hydrolysate derivatives, by which are meant protein components which, because of their chemical constitution, form particular three-dimensional spatial structures which are familiar to the skilled worker from protein chemistry under the names secondary, tertiary or else quaternary structure.

For the preparation of the composite material, reference is expressly made to the disclosure in WO 01/01930 A1.

The content of the composite material in the oral and dental hygiene products of the invention is from 0.01 to 10% by weight, preferably 0.1 to 2% by weight, based on the total weight of the product.

The oral and dental hygiene products of the invention further comprise from 10 to 35% by weight, in particular 10 to 25% by weight, of a cleaning body mixture, of which from 0.01 to 5% by weight of the cleaning body mixture consist of the polishing agent aluminum oxide.

Cleaning agents are among the essential ingredients of a toothpaste and are present, depending on their intended function, alone or in combination with other cleaning bodies. They serve to remove mechanically the uncalcified dental plaque and should ideally lead to shining of the tooth surface (polishing effect) with a simultaneous minimal scouring effect (abrasive effect) and damage to the enamel and the dentin.

The abrasive behavior of cleaning bodies is substantially determined by their hardness, particle size distribution and surface structure. Consequently, in the selection of suitable cleaning bodies, in particular those which have a minimal abrasive effect together with a high cleaning efficiency will preferably be selected.

The substances currently used as cleaning bodies are those having small particle sizes, being substantially free of sharp corners and edges and having a suitable hardness.

Since the deposition of the composite material is to be ensured and is to remain after the cleaning process as well, further cleaning agents specifically selected are those which have particularly low abrasiveness and good cleaning action. Suitable further cleaning agents within the meaning of the invention therefore have an average particle size of 1-200 μm, preferably 1-50 μm and especially 1-10 μm.

The cleaning agents of the invention can in principle be selected from silicas, aluminum hydroxide, aluminum oxide, silicates, organic polymers or mixtures thereof. However, the products of the invention may also comprise so-called metaphosphates, alkaline earth metal carbonates or bicarbonates and calcium-containing polishing components.

It may be preferred according to the invention to employ silicas as cleaning agents in toothpastes or liquid dental cleaning compositions. Among the silica cleaning agents, a distinction is made in principle between gel silicas, hydrogel silicas and precipitated silicas. Precipitated and gel silicas are particularly preferred according to the invention because wide variation in fluoride active substances is particularly good. They are moreover also particularly suitable for producing gel or liquid toothpastes.

Gel silicas are generated by reacting sodium silicate solutions with strong aqueous mineral acids to form a hydrosol, aging to give the hydrogel, washing and subsequent drying. Drying under mild conditions to water contents of from 15 to 35% by weight results in so-called hydrogel silicas as are also described for example in U.S. Pat. No. 4,153,680. Drying of this hydrogel silica to water contents of below 15% by weight leads to irreversible shrinkage of the previously loose structure to the dense structure of the so-called xerogel. Such xerogel silicas are disclosed for example in U.S. Pat. No. 3,538,230.

A second, preferably suitable group of silica polishing agents are the precipitated silicas. These are obtained by precipitating silica from dilute alkali metal silicate solutions by adding strong acids under conditions with which aggregation to the sol and gel cannot occur. Suitable methods for preparing precipitated silicas are described for example in DE-A 25 22 586 and DE-A 31 14493. Particularly suitable according to the invention is a precipitated silica prepared as disclosed in DE-A 31 14 493 and having a BET surface area of 15-110 m²/g, a particle size of from 0.5 to 20 μm, with the specification that at least 80% by weight of the primary particles are below 5 μm, and a viscosity in 30% strength glycerol-water (1:1) dispersion of 30-60 Pa s (20° C.) in an amount of 10-20% by weight of the toothpaste. Preferably suitable precipitated silicas of this type additionally have rounded corners and edges and can be obtained for example under the proprietary name Sident® 12 DS from Degussa.

Further precipitated silicas of this type are Sident® 8 from Degussa and Sorbosil® AC 39 from Crosfield Chemicals. These silicas are distinguished by a smaller thickening effect and a somewhat larger average particle size of 8-14 μm with a specific surface area of 40-75 m²/g (BET) and are particularly suitable for liquid toothpastes. These ought to have a viscosity (25° C., rate of shear D=10 s⁻¹) of 10-100 Pa s.

It is additionally possible to employ the silicas of Zeodent® type from Huber-Corp., Tixosil® from Rhodia and other Sorbosil types in the products of the invention. Zeodent® 113, Tixosil® 123 and 73 and Sorbosil® AC39 are particularly preferred.

Toothpastes having a higher viscosity of more than 100 Pa s (25° C., D=10 s⁻¹) by contrast require a sufficiently large proportion of silicas having a particle size of less than 5 μm, preferably at least 3% by weight of a silica having a particle size of from 1 to 3 μm. Besides said precipitated silicas, also added to such toothpastes are so-called thickening silicas which have finer particles and a BET surface area of 150-250 m²/g. Examples of commercial products which may be mentioned as complying with the stated conditions are in particular Sipernat® 22 LS or Sipernat® 320 DS from Degussa.

A suitable and preferred aluminum oxide polishing agent is a slightly calcined alumina having a content of α- and γ-aluminum oxide in an amount of about 0.01 to 5% by weight, preferably 0.1 to 2% by weight, based on the total weight of the product.

Suitable slightly calcined aluminas are prepared from aluminum hydroxide by calcination. Aluminum hydroxide is converted by calcination into α-Al₂O₃ which is thermodynamically stable at temperatures above 1200° C. The thermodynamically unstable Al₂O₃ modifications which occur at temperatures between 400 and 1000° C. are referred to as gamma forms (cf. Ullmann, Enzyclopädie der technischen Chemie, 4th edition (1974), Volume 7, page 298). The degree of calcination, i.e. the conversion into the thermodyamically stable α-Al₂O₃, can be set at any level through the choice of the temperature and the duration of the calcination. Slight calcination results in an alumina with a γ-Al₂O₃ content which is lower when the chosen calcination temperature is higher and the chosen duration of calcination is longer. Slightly calcined aluminas differ from pure γ-Al₂O₃ by the agglomerates being less hard, the specific area being larger and the pore volumes being larger.

The dentin abrasion (RDA) of the slightly calcined aluminas to be used according to the invention having a proportion of 10-50% by weight of γ-Al₂O₃ is only 30-60% of the dentin abrasion of a highly calcined, pure α-Al₂O₃ (measured in a standard toothpaste with 20% by weight alumina as only polishing agent).

Aluminum oxide polishing agents of various degrees of calcination, fineness of grinding and bulk densities are commercially available, e.g. the “Poliertonerden” from Giulini-Chemie or ALCOA. A preferably suitable type “Poliertonerde P10 feinst” has an agglomerate size of below 20 μm, an average primary crystallite size of 0.5-1.5 μm and a bulk density of 500-600 g/l.

The use of silicates as polishing agent components may likewise be preferred according to the invention. They are employed in particular as cleaning bodies in modern practice. Examples of silicates which can be employed according to the invention are aluminum silicates and zirconium silicates. The sodium aluminum silicate of the empirical formula Na₁₂(AlO₂)₁₂(SiO₂)₁₂×7H₂O may be particularly suitable as polishing agent, such as, for example, the synthetic zeolite A.

Examples of water-insoluble metaphosphates of the invention are in particular sodium metaphosphate, calcium phosphate such as, for example, tricalcium phosphate, calcium hydrogen phosphate, calcium hydrogen phosphate dihydrate and calcium pyrophosphate.

A further possibility according to the invention is to employ magnesium carbonate, magnesium hydrogen phosphate, trimagnesium phosphate or sodium hydrogen carbonate as polishing agents, especially mixed with other polishing agents.

A further polishing agent which is suitable for use in the oral and dental hygiene products of the invention is calcium phosphate dihydrate (CaHPO₄×2H₂O). Calcium phosphate dihydrate occurs naturally as brushite and is obtainable commercially in suitable particle sizes of from 1 to 50 μm as polishing agent.

The total RDA of the oral and dental hygiene product is according to the invention from 50 to 170, in particular 60 to 120.

The specific adjustment of the RDA for the oral and dental hygiene products of the invention ensures a gentle but efficient cleaning of the mouth, it being possible that a smaller scratching effect on the hard tooth substance (enamel) is achieved. Attention must additionally be given to the abrasiveness of a dental cleaning composition on the dentin, since this is distinctly softer than the enamel and may, especially when the necks of teeth are exposed, lead to tenderness. The RDA can therefore be cited as characteristic of the gentleness of a dental cleaning composition. An RDA was adjusted according the invention, through suitable choice of the cleaning body mixture, with which gentle but thorough cleaning of the teeth is possible but which nevertheless does not stand in the way of simultaneous deposition of remineralizing component.

The RDA is determined by the method of Hefferren, Journal of Dental Research, July-August (1976), page 563-573; U.S. Pat. No. 4,340,583; U.S. Pat. No. 4,420,312 and U.S. Pat. No. 4,421,527. This entails radiolabeling of the test tooth by electron irradiation, subsequent brushing with a defined toothpaste slurry, measuring the radioactivity of the abraded tooth material, and comparing with the RDA of a standard toothpaste.

It may be preferred according to the invention for the remineralization process brought about by the composite material to be assisted further by adding a remineralization-promoting agent. Remineralization-promoting agents of this type are usually admixed with the oral and dental hygiene products in amounts of from 0.1 to 10% by weight, preferably 0.1 to 5% by weight and in particular 0.1 to 3% by weight, in each case based on the total weight of the product.

The remineralization-promoting component in the products of the invention promotes the remineralization of the enamel and the sealing of dental lesions and is selected from fluorides, microparticulate phosphate salts of calcium such as, for example, calcium glycerol phosphate, calcium hydrogen phosphate, hydroxyapatite, fluoroapatite, F-doped hydroxyapatite, dicalcium phosphate dihydrate and calcium fluoride. However, magnesium salts such as, for example, magnesium sulfate, magnesium fluoride or magnesium monofluorophosphate also have remineralizing effects.

In a preferred embodiment of the invention, a magnesium salt is employed as remineralization-promoting agent.

Suitable embodiments of the oral and dental hygiene products of the invention are solid, liquid or semiliquid toothpastes and tooth gels.

The oral and dental hygiene products of the invention comprise in a further preferred embodiment additional toothpaste ingredients such as surfactants, humectants, binders, flavorings and active substances to counter dental and gingival disorders.

To improve the cleaning effect and the foam formation by the oral and dental hygiene products of the invention, normally surface-active surfactants or surfactant mixtures are employed. They promote rapid and complete dissolution and distribution of toothpastes in the mouth and simultaneously assist the mechanical removal of dental plaque, especially at places difficult to access by a toothbrush. In addition, they favor the incorporation of water-insoluble substances, for example of aromatic oils, stabilize the polishing agent dispersion and assist the anticaries effect of fluorides.

It is possible in principle to use anionic surfactants, zwitterionic and ampholytic surfactants, nonionic surfactants, cationic surfactants or mixtures of these compounds as surfactants in toothpaste formulations. Toothpastes preferably comprise according to the invention at least one surfactant from the group of anionic surfactants.

The surfactant or the surfactant mixture is normally employed in the compositions of the invention in an amount of 0.1-10% by weight, preferably 0.3-7% by weight and in particular 1-5% by weight, based on the total weight of the composition.

Anionic Surfactants

Suitable surfactants with a good foaming effect are anionic surfactants which also have a certain enzyme-inhibiting effect on the bacterial metabolism of the dental plaque.

These include for example alkali metal or ammonium salts, especially sodium salts, of C₈-C₁₈-alkanecarboxylic acids, of alkyl polyglycol ether sulfates having 12-16 C atoms in the linear alkyl group and 2-6 glycol ether groups in the molecule, of linear alkane-(C₁₂-C₁₈)-sulfonates, sulfosuccinic acid monoalkyl(C₁₂-C₁₈)esters, sulfated fatty acid monoglycerides, sulfated fatty acid alkanolamides, sulfoacetic acid alkyl(C₁₂-C₁₆)esters, acylsarcosines, acyl taurides and acylisethionates having in each case 8-18 C atoms in the acyl group.

It is preferred to use at least one anionic surfactant, in particular a sodium laurylsulfate having 12-18 C atoms in the alkyl group. One surfactant of this type is sodium lauryl sulfate which is commercially available for example under the name Texapon® K12G.

Zwitterionic and Ampholytic Surfactants

It may be preferred according to the invention to employ zwitterionic and/or ampholytic surfactants, preferably in combination with anionic surfactants. Surface-active compounds which have at least one quaternary ammonium group and at least one carboxylate and one sulfonate group in the molecule are referred to as zwitterionic surfactants. Particularly suitable zwitterionic surfactants are the so-called betaines such as the N-alkyl-N,N-dimethylammonium glycinates, for example trimethylammonium glycinate, cocoalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines having in each case 8 to 18 C atoms in the alkyl or acyl group, and cocoacylaminoethylhydroxyethylcarboxymethyl glycinate. The fatty amide derivative known under the CTFA name cocamidopropyl betaine is particularly preferred. Such products are commercially available for example under the name Tego-Betain® BL 215 and ZF 50, and Genagen® CAB.

Ampholytic surfactants mean those surface-active compounds which, apart from a C₈-C₁₈-alkyl or acyl group in the molecule, comprise at least one free amino group and at least one —COOH or —SO₃H group and are able to form inner salts. Examples of suitable ampholytic surfactants are N-alkylglycines, N-alkylpropionic acids, N-alkylaminobutyric acids, N-alkyl-imidodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 C atoms in the alkyl group. Particularly preferred ampholytic surfactants are N-cocoalkylaminopropionate, cocoacylaminoethylaminopropionate and C₁₂-C₁₈-acylsarcosine. Also suitable besides the ampholytic are quaternary emulsifiers, with particular preference for those of the ester quat type, preferably methyl-quaternized di-fatty acid triethanolamine ester salts.

Nonionic Surfactants

Nonionic surfactants are particularly suitable according to the invention for assisting the cleaning effect. Particularly preferred nonionic surfactants are those selected from at least one of the following groups:
adducts of 2 to 30 mol of ethylene oxide and/or 0 to 5 mol of propylene oxide with linear fatty alcohols having 8 to 22 C atoms, with fatty acids having 12 to 22 C atoms and with alkylphenols having 8 to 15 C atoms in the alkyl group;
C₁₂-C₁₈ fatty acid mono- and diesters of adducts of 1 to 30 mol of ethylene oxide with glycerol;
glycerol mono- and diesters and sorbitan mono- and diesters of saturated and unsaturated fatty acids having 6 to 22 carbon atoms and their ethylene oxide adducts;
alkyl mono- and oligoglycosides having 8 to 22 carbon atoms in the alkyl radical and their ethoxylated analogs;
adducts of 15 to 60 mol of ethylene oxide with castor oil and/or hardened castor oil;
polyol esters and especially polyglycerol esters such as, for example, polyglycerol polyricinoleate, polyglycerol poly-12-hydroxystearate or polyglycerol dimerate.

Likewise suitable are mixtures of compounds from a plurality of these substance classes;
adducts of 2 to 15 mol of ethylene oxide with castor oil and/or hardened castor oil;
partial esters based on linear, branched, unsaturated or saturated C₆-C₂₂ fatty acids, ricinoleic acid and 12-hydroxystearic acid and glycerol, polyglycerol, pentaerythritol, dipentaerythritol, sugar alcohols (e.g. sorbitol), sucrose, alkyl glucosides (e.g. methyl glucoside, butyl glucoside, lauryl glucoside) and polyglucosides (e.g. cellulose);
mono-, di- and trialkyl phosphates, and mono-, di- and/or tri-PEGalkyl phosphates and their salts;
wool wax alcohols;
polysiloxane-polyalkyl-polyether copolymers and corresponding derivatives;
mixed esters of pentaerythritol, fatty acids, citric acid and fatty alcohol as disclosed in DE 1165574 and/or mixed esters of fatty acids having 6 to 22 carbon atoms, methylglucose and polyols, preferably glycerol and polyglycerol and
polyalkylene glycols.

The adducts of ethylene oxide and/or propylene oxide with fatty alcohols, fatty acids, alkylphenols, glycerol mono- and diesters and sorbitan mono- and diesters of fatty acids or with castor oil are known commercially available products and are preferred according to the invention. They are mixtures of homologs whose average degree of alkoxylation corresponds to the ratio of the amounts of substance of ethylene oxide and/or propylene oxide and substrate with which the addition reaction is carried out. C₁₂-C₁₈ fatty acid mono- and diesters of adducts of ethylene oxide with glycerol are disclosed in DE 2024051 as refatting agents for cosmetic preparations.

C₈-C₁₈-alkyl mono- and oligoglycosides, their preparation and their use are known in the prior art, for example from U.S. Pat. No. 3,839,318, DE-A-20 36 472, EP-A-77 167 or WO-A-93/10132. Their preparation takes place in particular by reacting glucose or oligosaccharides with primary alcohols having 8 to 18 C atoms. In respect of the glycoside residue, both monoglycosides in which a cyclic sugar residue is glycosidically linked to the fatty alcohol, and oligomeric glycosides having a degree of oligomerization up to preferably about 8 are suitable. In this connection, the degree of oligomerization is a statistical average based on the homolog distribution usual for such industrial products. A suitable and preferred alkyl (oligo)glycoside is an alkyl (oligo)glycoside of the formula RO(C₆H₁₀O)ₓ—H in which R is an alkyl group having 12 to 14 C atoms, and x has an average value of from 1 to 4.

A particularly preferred example which should be mentioned of a nonionic surfactant which can be employed according to the invention is for example PEG-glyceryl stearate which is commercially available under the name Tagat® S.

Humectants are normally employed in dental cosmetics to prevent desiccation and to control the consistency and low-temperature stability of the products. They may, however, also serve to promote suspension and to influence the taste or gloss.

Humectants ordinarily used are toxicologically acceptable polyols such as for example sorbitol, xylitol, glycerol, mannitol, 1,2-propylene glycol or mixtures thereof; however, polyethylene glycols having molecular weights of 400-2000 may also serve as humectants in toothpastes.

It is preferred to combine a plurality of humectant components, in which case the combination of glycerol and sorbitol with a content of 1,2-propylene glycol or polethylene glycol is to be regarded as particularly preferred.

Depending on the product type, the humectant or the mixture of humectants is present in the overall composition in an amount of 10-85% by weight, preferably 15-70% by weight and in particular 25-50% by weight.

The products of the invention additionally comprise in a preferred embodiment at least one binder or thickener. These act to control the consistency and in addition prevent the separation of the liquid and solid ingredients.

The amounts thereof employed in the compositions of the invention are 0.1-5% by weight, preferably 0.1-3% by weight and in particular 0.5-2% by weight.

Examples used according to the invention are natural and/or synthetic water-soluble polymers such as alginates, carrageenans, agar-agar, guar gum, gum arabic, succinoglycan gum, guar flour, carob flour, tragacanth, karaya gum, xanthan, pectins, cellulose and their ionic and nonionic derivatives such as, for example, carboxymethylcellulose, hydroxyethylcellulose or methylhydroxypropylcellulose, hydrophobically modified celluloses, starch and starch ethers.

Also used as binders or thickeners are water-soluble carboxyvinyl polymers (e.g. Carbopol® types), polyvinyl alcohol, polyvinylpyrrolidone and high molecular weight polyethylene glycols (especially those with molecular weights of 10²-10⁶ D). This function can likewise be fulfilled by sheet silicates and fine-particle silicas (aerogel silicas and pyrogenic silicas).

In a further preferred embodiment, the oral and dental hygiene product of the invention comprises additional active substances to prevent dental and gingival disorders. Such active substances mean according to the invention anticaries active substances, antimicrobial active substances, tartar inhibitors, flavorings or any combination of these substances.

Anticaries Active Substances

Particularly suitable for controlling and preventing caries are fluorine compounds, preferably from the group of fluorides or monofluorophosphates in an amount of 0.1-0.5% by weight fluorine. Suitable fluorine compounds are, for example, sodium fluoride, potassium fluoride, tin fluoride, disodium monofluorophosphate (Na₂PO₃F), dipotassium monofluorophosphate or the fluoride of an organic amino compound.

Antimicrobial Active Substances

Examples of suitable antimicrobial component are phenols, resorcinols, bisphenols, salicylanilides and -amides and their halogenated derivatives, halogenated carbanilides and p-hydroxybenzoic esters.

Particularly suitable antimicrobial components are those which inhibit the growth of plaque bacteria. Suitable examples of antimicrobial active substances are halogenated diphenyl ethers such as 2,4-dichloro-2′-hydroxydiphenyl ether, 4,4′-dichloro-2′-hydroxydiphenyl ether, 2,4,4′-tribromo-2′-hydroxydiphenyl ether, 2,4,4′-trichloro-2′-hydroxydiphenyl ether (triclosan). Besides bromochlorophene, bisbiguanides such as chlorhexidine and alexidine, phenylsalicylic esters and 5-amino-1,3-bis(2-ethylhexyl)hexahydro-5-methylpyrimidine (hexetidine), also zinc and copper ions have an antimicrobial effect, with synergistic effects occurring in particular in combination with hexetidine and triclosan. It is also possible to employ quaternary ammonium compounds such as, for example, cetylpyridinium chloride, benzalkonium chloride, domiphene bromide and dequalinium chloride. Octapinol, octenidine and sanguinarine have also proved to have antimicrobial activity.

The antimicrobial active substances are preferably employed in amounts of 0.01-1% by weight in the products of the invention. It is particularly preferred to use Irgacare® MP in an amount of 0.01-0.3% by weight.

Tartar Inhibitors

Tartar comprises mineral deposits which are very similar to natural enamel. In order to inhibit tartar formation, substances which intervene specifically in crystal nucleation and prevent nuclei which are already present from growing further are added to the dental cleaning compositions of the invention. Examples thereof are condensed phosphates which are preferably chosen from the group of tripolyphosphates, of pyrophosphates, of trimetaphosphates or mixtures thereof. They are employed in the form of their alkali metal or ammonium salts, preferably in the form of their sodium or potassium salts. Aqueous solutions of these phosphates typically have an alkaline reaction, so that the pH of the dental hygiene products of the invention is adjusted where appropriate to values of 7.5-9 by adding acid. Examples of acids which can be used in this connection are citric acid, phosphoric acid or acidic salts, e.g. NaH₂PO₄. The desired pH of the dental hygiene product can, however, also be adjusted by adding acidic salts of the condensed phosphates, e.g. K₂H₂P₂O₇.

It is also possible to employ according to the invention mixtures of various condensed phosphates and/or hydrated salts of the condensed phosphates. Tartar inhibitors are normally employed in amounts of 0.1-5% by weight, preferably 0.1-3% by weight and in particular 0.1-2% by weight in the products of the invention.

Further suitable tartar inhibitors are organophosphonates such as 1-azacycloheptane-2,2-diphosphonate (Na salt), 1-hydroxyethane-1,1-diphosphonate (Na salt) and zinc citrate.

Active Substances to Counter Hypersensitive Teeth

The products of the invention preferably further comprise active substances to counter hypersensitive teeth, they are selected from potassium and strontium salts such as potassium chloride, potassium sulfate, potassium bicarbonate, potassium citrate, potassium acetate, potassium nitrate, strontium chloride, strontium nitrate, strontium citrate, strontium acetate and strontium lactate and eugenol.

The eugenol may be present mixed with aromatic oils in the oral and dental hygiene products. It is preferably present in the compositions in the form of clove bud oil.

The oral and dental hygiene products of the invention preferably comprise at least 0.5% by weight of potassium or strontium ions in the form of a dissolved salt and at least 0.01% by weight of eugenol in pure form or in the form of the clove bud oil.

Flavorings

The products of the invention preferably comprise flavorings, which include, for example, sweeteners and/or aromatic oils.

Examples of suitable sweeteners are saccharinates (especially sodium saccharinate), cyclamates (especially sodium cyclamate) and sucrose, lactose, maltose or fructose.

Suitable aromatic oils are all natural and synthetic aromas used for oral and dental hygiene products. Natural aromas can be used both in the form of the essential oils (mixture) isolated from the herbs and in the form of the individual components isolated therefrom. At least one aromatic oil from the group of peppermint oil, spearmint oil, anise oil, star anise oil, carraway oil, eucalyptus oil, fennel oil, cinnamon oil, clove oil, geranium oil, sage oil, pimento oil, thyme oil, marjoram oil, basil oil, citrus oil, gaultheria oil or one or more than one of the components of these oils isolated therefrom or produced synthetically ought preferably to be present. The principal components of said oils are, for example, menthol, carvone, anethol, cineol, eugenol, cinnamaldehyde, caryophyllene, geraniol, citronellol, linalool, salvene, thymol, terpinene, terpinol, methylchavicol and methyl salicylate. Further suitable aromas are, for example, menthyl acetate, vanillin, ionones, linalyl acetate, rhodinol and piperitone.

Finally, further customary aids can be present to improve the stability and the organoleptic properties of the oral and dental hygiene products. Examples of such aids are:
vitamins, e,g. retinol, biotin, tocopherol, ascorbic acid and derivatives thereof (e.g. esters, salts);
pigments, e.g. titanium dioxide or zinc oxide;
colored pigment particles, for example colored silica particles like those commercially available for example under the proprietary name Sorbosil® BFG 51, BFG 52 and BFG 53 or Sorbosil® 2352. It is also possible to use mixtures of differently colored pigment particles. Gel silica particles with, for example, a strong orange, red or blue color can be present in the products of the invention in amounts of 0.1-1.0% by weight;
bleaches such as, for example, hydrogen peroxide and hydrogen peroxide precursors;
colorants;
pH-adjusting agents and buffer substances, e.g. sodium citrate, sodium bicarbonate or potassium and sodium phosphates;
preservatives, e.g. methyl, ethyl or propyl p-hydroxybenzoate, sodium sorbate, sodium benzoate, bromochlorophene or triclosan;
wound-healing and antiinflammatory substances such as, for example, allantoin, urea, panthenol, azulene or chamomile extract, acetylsalicylic acid derivatives, alkali metal thiocyanates;
mineral salts such as zinc, magnesium and manganese salts, for example sulfates.

All optional toothpaste ingredients are present together in an amount of about 2 to 10% by weight based on the total weight in the products of the invention.

A second aspect of the invention is the cosmetic use of the of an oral and dental cleaning composition of the invention for the prevention and control of hypersensitive teeth.

Cosmetic use means the non-therapeutic use of the oral and dental cleaning composition of the invention for daily cleaning and care of the teeth and the mouth.

A third aspect of the invention is the cosmetic use of the oral and dental hygiene product of the invention for brightening the teeth and for preventing discolorations and rediscolorations.

The brightening of teeth is determined by comparing teeth treated with the dental cleaning composition of the invention and teeth treated with a comparative formulation, using a commercially available colorimeter (from Lange).

A fourth aspect of the invention is the cosmetic use of the oral and dental hygiene product of the invention for preventing adhesion of plaque and reduction, associated therewith, in formation of new plaque on tooth surfaces. It is possible thereby to reduce the plaque adhesion, which is promoted by faults, on the tooth surface.

A fifth aspect of the invention is the cosmetic use of the oral and dental hygiene product of the invention for remineralization of dental lesions.

The effect of the composite material can be promoted by combination with a suitable toothbrush. The cleaning efficiency of a composite-containing dental hygiene product is possible, with high deposition, through the choice of a suitable bristle arrangement and of a suitable design of the brush, especially of the brushhead and of the cut.

Suitable packaging for the dental hygiene preparations comprising composite materials of the invention comprises tubes and dispensers, pumps and other packagings which facilitate dosage.

The following examples are intended to explain the subject matter of the invention in detail without restricting it thereto.

The stated amounts used are based, unless indicated otherwise, on % by weight.

### EXAMPLES

### 1) Example of a Semiviscous Toothpaste Preparation

| | |
|---|---|
| Neosorb ® 70/70¹ | 19.000 |
| Glycerol 86% pure | 18.000 |
| Poliertonerde ® P10 feinst² | 0.500 |
| Sident ® 8³ | 14.000 |
| zacyclohetane-2,2-diphosphonic acid | 0.500 |
| NaOH | 0.158 |
| Sodium sacchariate | 0.200 |
| Sodium fluoride | 0.100 |
| PHB methyl ester | 0.100 |
| Disodium phosphate | 0.100 |
| Trisodium phosphate | 0.050 |
| Nanit in glyceric dispersion⁴ | 0.855 |
| Keltrol ® F⁵ | 1.250 |
| 1,2-Propylene glycol | 5.000 |
| Tego Betain ® BL 215⁶ | 0.600 |
| Texapon ® K12 G⁷ | 1.500 |
| Aroma | 1.000 |
| Water | ad 100 |

### 2) Example of a Low-viscosity Toothpaste Preparation

| | |
|---|---|
| Neosorb ® 70/70 | 55.000 |
| Sident ® 8 | 12.000 |
| Poliertonerde ® P10 | 0.500 |
| Disodium phosphate | 0.200 |
| Sodium fluoride | 0.320 |
| Sodium saccharinate | 0.200 |
| Sodium benzoate | 0.490 |
| Zinc sulfate-7H20 | 0.088 |
| Titanium dioxide | 1.000 |
| Nanit in glyceric dispersion | 0.855 |
| Polywachs ® 1550 | 1.000 |
| Keltrol ® F | 0.500 |
| Texapon ® K 1296⁸ | 1.500 |
| Tagat ® S⁹ | 0.500 |
| Tego Betain BL 215 | 0.600 |
| NaOH | 0.316 |
| Ethanol | 2.000 |
| Aroma | 1.200 |
| Water | ad 100 |

### 3) Example of a Viscous (Solid) Toothpaste

| | |
|---|---|
| Neosorb ® 70/70 | 30.000 |
| Poliertonerde P10 | 1.000 |
| Phosphoric acid 85% pure | 0.050 |
| Sident ® 8 | 12.000 |
| Sident ® 22 S¹⁰ | 8.000 |
| Sipernat ® (FK) 320DS¹¹ | 1.000 |
| Titanium dioxide | 1.000 |
| Nanit in glyceric dispersion | 0.855 |
| Disodium phosphate | 0.200 |
| Sodium fluoride | 0.320 |
| Sodium saccharinate | 0.200 |
| NaOH | 0.158 |
| Polydiol ® 400¹² | 3.000 |
| Cekol ® 2000 H¹³ | 1.000 |
| Texapon ® K12 G | 1.350 |
| Aroma | 1.000 |
| Water | ad 100 |

It was demonstrated in experiments that the deposition effect is retained even with use of polishing agents such as aluminum hydroxides. These experiments were carried out for example with samples of dentin, and the growth of hydroxyapatite was assessed.

The following commercial products were used:
1 Neosorb®: INCI: Sorbitol; about 70% in water; manufacturer: Roquette
2 Poliertonerde P10 feinst: INCI: alumina manufacturer: Alcoa
3 Sident® 8: INCI: hydrated silica; manufacturer: Degussa
4 Nanit in glyceric dispersion: composite material of the invention as 10% strength dispersion in glycerol
5 Keltrol® F: INCI: xanthan gum manufacturer: CP Kelco
6 Tego Betain® BL 215: INCI: cocamidopropyl betaine; AS: 30% manufacturer: Goldschmidt
7 Texapon® K12G: INCI: sodium lauryl sulfate; AS. 95-99% manufacturer: Cognis
8 Texapon® K1296: sodium lauryl sulfate; AS: 90% manufacturer: Cognis
9 Tagat® S: INCI: PEG-30 glyceryl stearate manufacturer: Tego Cosmetics
10 Sident® 22 S: INCI: hydrated silica; manufacturer: Degussa
11 Sipernat® FK 320 DS: INCI: hydrated silica; manufacturer: Degussa
12 Polydiol® 400: INCI: polyethylene glycol; MW 380-420 manufacturer: Cognis
13 Cekol® 2000H: INCI: cellulose gum manufacturer: Noviant

1. An oral and dental hygiene product comprising
a) a composite material comprising
calcium salts which are slightly soluble in water, in the form of nanoparticulate primary particles having a length of from 5 to 150 nm and a cross section of from 2 to 50 nm and
protein components selected from the group consisting of proteins, protein hydrolysates and protein hydrolysate derivatives, and
b) 10 to 35% by weight of a cleaning agent mixture, characterized in that the cleaning agent mixture comprises from 0.01 to 5% by weight of aluminum oxide polishing agent.

2. The oral and dental hygiene product as claimed in claim 1, characterized in that it has a dental abrasion of from 50 to 170.

3. The oral and dental hygiene product as claimed in claim 1, characterized in that the slightly soluble calcium salts are selected from fluoroapatite and hydroxyapatite and mixtures thereof.

4. The oral and dental hygiene product as claimed in claim 1, characterized in that the protein component is selected from the group consisting of structure-forming proteins, collagen, gelatin, keratin, casein, wheat protein, rice protein, soybean protein, almond protein and their hydrolysates and hydrolysate derivatives.

5. The oral and dental hygiene product as claimed in claim 1, characterized in that the content of the composite material is from 0.01 to 10% by weight based on the total weight of the product.

6. The oral and dental hygiene product as claimed in claim 1, characterized in that the nanoparticulate primary particles are rod-like.

7. The oral and dental hygiene product as claimed in claim 1, characterized in that the nanoparticulate primary particles form rod-like crystals having length-to-breadth ratio of 3 to ≦5.

8. The oral and dental hygiene product as claimed in of claim 1, characterized in that the cleaning agent is selected from the group consisting of silicas, aluminum oxides, silicates, metaphosphates and alkaline earth metal carbonates and bicarbonates.

9. The oral and dental hygiene product as claimed in claims 1, characterized in that the product further comprises at least one remineralization-promoting component.

10. The oral and dental hygiene product as claimed in claim 9, characterized in that the remineralization-promoting component is selected from magnesium salts.

11. The oral and dental hygiene product as claimed in claim 1, characterized in that it comprises as further toothpaste ingredients one or more of surfactants, humectants, binders and active substances that counter dental and gingival disorders.

12. The oral and dental hygiene product as claimed in claim 1, characterized in that the active substances are selected from the group consisting of anticaries active substances, antimicrobial substances, tartar inhibitors, active substances to that counter hypersensitive teeth and mixtures of these substances.

13-16. (canceled)

17. The oral and dental hygiene product as claimed in claim 1, wherein the calcium salts consist of hydroxyapatite and the protein components consist of Type A gelatin.

18. An oral and dental hygiene product comprising
a) a composite material comprising
hydroxyapatite, in the form of nanoparticulate primary particles having a length of from 5 to 150 nm and a cross section of from 2 to 50 nm,
gelatin, and
b) 10 to 35% by weight of a cleaning agent mixture, characterized in that the cleaning agent mixture comprises from 0.01 to 5% by weight of aluminum oxide polishing agent.

19. The process for cleaning and remineralizing of teeth comprising the step of applying to teeth an oral and dental hygiene product comprising
a) a composite material comprising
calcium salts which are slightly soluble in water, in the form of nanoparticulate primary particles having a length of from 5 to 150 nm and a cross section of from 2 to 50 nm and
protein components selected from the group consisting of proteins, protein hydrolysates and protein hydrolysate derivatives, and
b) 10 to 35% by weight of a cleaning agent mixture, characterized in that the cleaning agent mixture comprises from 0.01 to 5% by weight of aluminum oxide polishing agent.

20. The process for the cleaning of sensitive teeth comprising the step of applying to the teeth the oral and dental hygiene product as claimed in claim 1.

21. The process for the prevention and control of hypersensitivity in teeth comprising the step of applying to the teeth the oral and dental hygiene product as claimed in claim 1.

22. The process for brightening teeth and preventing discolorations and re-discolorations, comprising the step of applying to the teeth the oral and dental hygiene product as claimed in claim 1.

23. The process for preventing adhesion of plaque to teeth comprising the step of applying to the teeth the oral and dental hygiene product of claim 1.

24. The process for remineralizing of dental lesions in teeth comprising the step of applying to the teeth the oral and dental hygiene product of claim 1.
